(19) United States
(12) Patent Application Publication
(10) Pub. No.: US 2007/0221529 A1
(43) Pub. Date: Sep. 27, 2007

(54) Packet containing moistened towelette used for cleaning pruning tools

(76) Inventors: Marlene Gioia-Smickle, New York, NY (US); Marta Lynne Smickle, Dingmans Ferry, PA (US)

Correspondence Address: Marlene Gioia Smickle, 11721 Pocono Mt. Lake Forest, Dingmans Ferry, PA, 18328, US

(21) Appl. No.: 11/728,444

(22) Filed: Mar. 26, 2007

Related U.S. Application Data
(60) Provisional application No. 60/786,065, filed on Mar. 27, 2006.

(51) Int. Cl. B65D 73/00; B65D 81/24

(52) U.S. Cl. 206/494; 206/484; 206/812; 206/205

(57) ABSTRACT

Disclosed is a system and method for tool maintenance. The packet includes a towelette having a fungicide, bactericide, or disinfectant. After the towelette is used on a pruning tool, it can be returned to the packet for easy disposal.

### CROSS-REFERENCE TO RELATED CASES

This application claims priority to U.S. Provisional Application No. 60/786,065, filed Mar. 27, 2006, the entirety of which is incorporated herein by reference.

### BACKGROUND OF THE INVENTION

### Field of the Invention

The present invention relates to tool maintenance and, more particularly, to a system and method for cleaning and disinfecting pruning tools.

Pruning tools used by arborists, landscape maintenance contractors, plant care professionals, or home gardeners, should be cleaned between each plant to be pruned and sometimes between each cut on a plant. Disease, bacteria and fungus (among other things) can be transmitted from plant to plant by the pruning tools.

It is common practice to dip pruning tools into a bucket or pail containing a cleaning solution, to spray the tools at the end of a work day or, cleaning the tool by dipping or spraying after working on a plant with an obvious problem. Unfortunately, the extent of some infections such as fire blight or botryodiplodia are not obvious. It is inconvenient and costly to stop between each pruning cut or between each plant being pruned to clean equipment when there isn't an obvious problem.

It is not convenient or practical to carry a spray can or a bucket of cleaning dip with you when pruning. In fact, a bucket of cleaning solution is potentially detrimental to landscaping, if spilled. Also, the dipping or spraying action does not remove the harmful organisms.

### SUMMARY OF THE INVENTION

It would be convenient and useful to have a wipe impregnated with a cleaning solution that can be carried in a pocket or tool sheath. The same technique needed to wash infectious organisms from your hands should be used to when cleaning pruning tools. Wiping the pruning tools after applying the cleaning solution is preferable to properly clean the pruning tools.

Disclosed is a packet containing a durable paper disposable towelette impregnated with a cleaning solution. The packet can be made of a material such as a foil, foil-lined paper, polymer-lined paper, Mylar, or another material. The packet is preferably made from heat-sealable laminates structures containing foil, polyethylene, polypropylene, polyester, paper, cellophane, or the like.

The packet is preferably opaque to prevent photo degradation of the cleaning solution. The cleaning solution can be any one or a combination of an antiseptic, antiviral, antibacterial, disinfectant, or other liquid used to kill harmful microorganisms, or the like.

The packet is preferably small enough to carry more than one and yet large enough to accommodate a towelette of a size sufficient to be handled easily while working. In a preferred embodiment, the unfolded towelette is 8″×10″. The packet and enclosed towelette are preferably disposable. In one embodiment, at least the towelette is biodegradable. In one embodiment, the packet is biodegradable.

The packet can be such that when opened along an indicated tear line, the packet forms a pouch to which the used towelette can be returned to and stored before disposal of the packet and towelette.

In another embodiment, the towelette is attached to packet along one edge. In another embodiment, the packet with towelette is disposable after use.

In one embodiment, minimum packet dimensions are 2″ (50.8 mm) wide×2″ (50.8 mm) high. In a preferred, the dimensions are 2.375″ (60 mm)×4.75″ (120 mm). In a further embodiment, the dimensions 8″ (203.2 mm) wide×9″ (228.6 mm) high. These dimensions can be varied depending on packaging convenience and the packets can be assembled in multiple “strings” with perforations or scoring in the packaging which would be used as a tear line separating each packet from the string of packets.

In another embodiment, the towelette is enforced with nylon strands, cotton strands, or the like. Additionally, in one embodiment, these strands extend beyond the perimeter of the towelette and are affixed to the towelette packet. In this manner, the towelette is affixed to the packet for easy disposal. In this manner, the packet stays with the towelette in an effort to prevent environmental pollution or dirtying a work area.

In one embodiment, the packet and towelette are reusable. The towelette can be a washable material. After use, the towelette can be reinserted into the packet. The towelette and packet can be soaked in a disinfecting solution. Afterwards, the packets can be reused with the disinfected towelettes. The packet can have an interlocking seal, an adhesive seal, or the like. In one embodiment, the towelette is in a sealed packet. In this embodiment, the towelette is not pre-moistened. The packet also has a separate pocket or capsule containing the disinfecting solution. Prior to use, the cell or capsule containing the disinfecting solution is ruptured thereby impregnating the towelette with the disinfecting solution.

### BRIEF DESCRIPTION OF THE FIGURES

FIG. 1 is a diagram of the invention according to a first embodiment;

FIG. 2 is a cross-section of the packet depicted in FIG. 1;

FIG. 3 is another embodiment of the package;

FIG. 4 shows another embodiment of the invention;

FIG. 5 is a cross-section of the embodiment shown in FIG. 4; and

FIG. 6 is another embodiment of the invention.

### DETAILED DESCRIPTION OF THE PREFERRED EMBODIMENTS

The present invention is described with respect to a pruning tool cleaner or sanitizer but may be applied to other industries and professions for cleaning, and sterilizing such as knives in the food service industry, scissors for hair stylists and hair dressers, clippers for stylists and groomers, and the like.

Pruning tools must be kept clean to avoid spreading diseases from one plant to another. Likewise, fungi can be spread from one plant to another via the pruning tools. Tree care workers, landscape contractors, arborists, and even weekend gardeners should follow pruning tool hygiene disciplines to prevent harm to the plants being pruned. Master Gardeners and home garden enthusiasts are taught to clean their tools after each use. Typically, instructors instruct people to dip tools in a bucket filled with cleaning solution and dry with a rag or rinse or spray tools with antiseptic spray and wipe with a rag. Blade cleaning needs to be performed between each cut when removing infected tissue. Blade cleaning must always be done between different plants. In order to effectively clean, wiping the cutting blade after spraying, rinsing, or dipping in said antiseptic/antibacterial/antifungal solution is vital. Unfortunately, it is not always practical to carry bucket of cleaning solution or a large can of spray antiseptic for all job sites. Further, it would be dangerous have these cleaning solutions while climbing trees or in other hazardous locations. Thus, many professionals wait until they are done for the day and sometimes forget to effectively clean their tools.

Disclosed is a convenient cleaning solution that can be carried in a pocket. The disclosed cleaning apparatus does not require special handling and is convenient for users. Further, the cleaning and wiping process is combined in a single step thereby simplifying the cleaning process. Further in one embodiment of the invention, a convenient and practical way of disposing the wipe is provided. The disclosed pre-moistened towelette is portable, convenient and durable. Finally, the disclosed packet containing a pre-moistened towelette is an effective way to insure properly maintained tools and healthy plants.

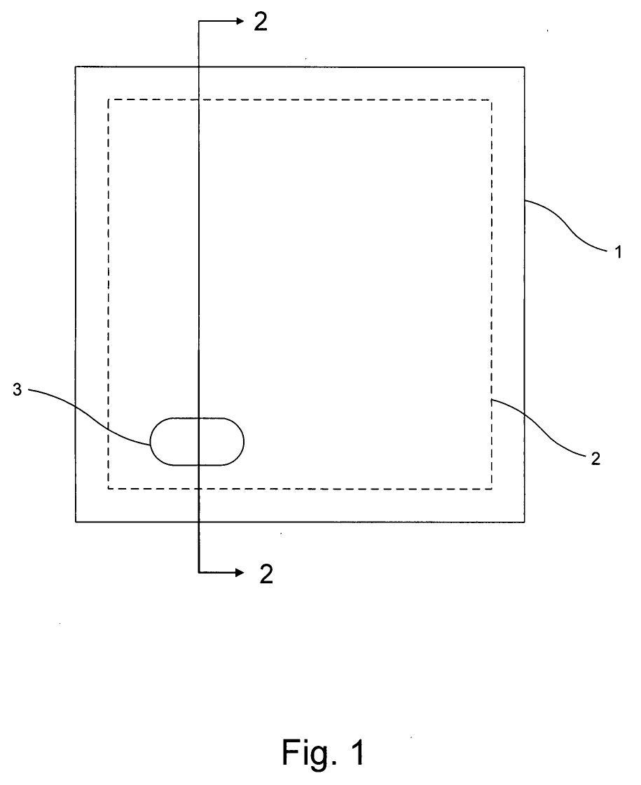

FIG. 1 depicts a first embodiment of the invention. As shown, a packet 1 contains a towel 2. The package 1 can be Mylar, plastic, foil, metalized paper, wax paper, or the like. Preferably, the package is opaque. The towelette is stored inside the package. In one embodiment, the towelette is folded to reduce the overall size of the package. The towelette can be paper, reinforced paper, linen, cloth, terry, sponge, or the like. Finally, the package contains a capsule 3. The capsule contains a disinfectant solution suitable for cleaning pruning tools. The disinfectant fluid is preferably alkyl dimethyl benzyl ammonium saccharinate and ethanol, sodium hypochlorite, o-phenylphenol o-benzyl-p-chlorophenol, quaternary ammonium chlorides, or the like. It should be noted that if multiple disinfectant solutions are required, the capsule can contain sections for the various disinfectant solutions or, multiple capsules can be present in a single package. Prior to use, the capsules are ruptured releasing the disinfecting liquid which is then soaked up into the towelette. It should be noted that in a preferable embodiment of the invention, the capsule is biodegradable such that if it is left outdoors, it will degrade over time without harming the environment.

In another embodiment of the invention, the towelette is pre-moistened such that no capsule need be present within the package. Alternatively, if required, the towelette can be moistened with a first liquid and a capsule can contain a second liquid or material used for disinfecting or other functions such as a fungicide or the like. It should be noted that while the term disinfectant is used throughout, the fluids can be fungicides, disinfectants, and/or bactericides. Additionally, the disinfectant can be oil based or suspended in an oil that simultaneously provides rust preventative maintenance to the pruning tool.

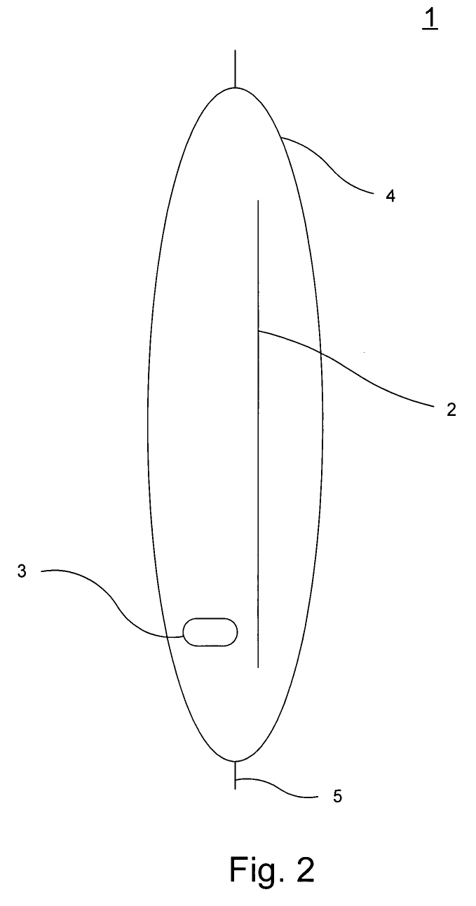

FIG. 2 shows a cross-section of the packet of FIG. 1. As shown, the packet 1 contains the towelette 2 and capsule 3. The edges of the packet 5 are sealed so that no fluid leaks out or air enters the packet. In one embodiment, the atmosphere inside the packet is inert. FIG. 2 shows one capsule 3. However, as noted above, multiple capsules containing various substances can be used. Alternatively, the towelette 2 can be pre-moistened or, the combination of the elements in capsule 3 combined with the material used to pre-moisten the towelette 2 provides the disinfecting and/or fungicidal result desired.

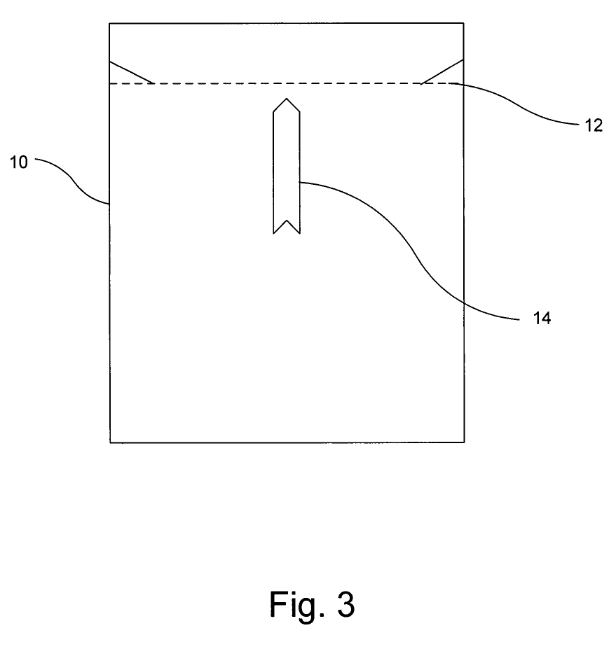

FIG. 3 shows another embodiment of the invention. As shown in FIG. 3, a packet 10 has a perforated and notched area 12. This area provides for easy opening of the packet. An adhesive strip 14 is provided such that the packet can be folded over and resealed. In this way, the towelette can be reused or, once the towelette is reused, it is placed inside the packet 10 for sealing and disposal.

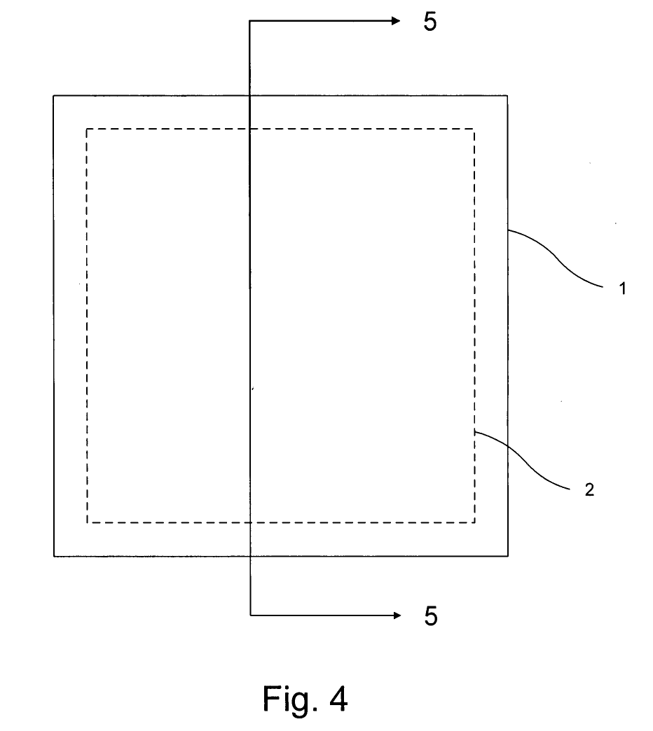

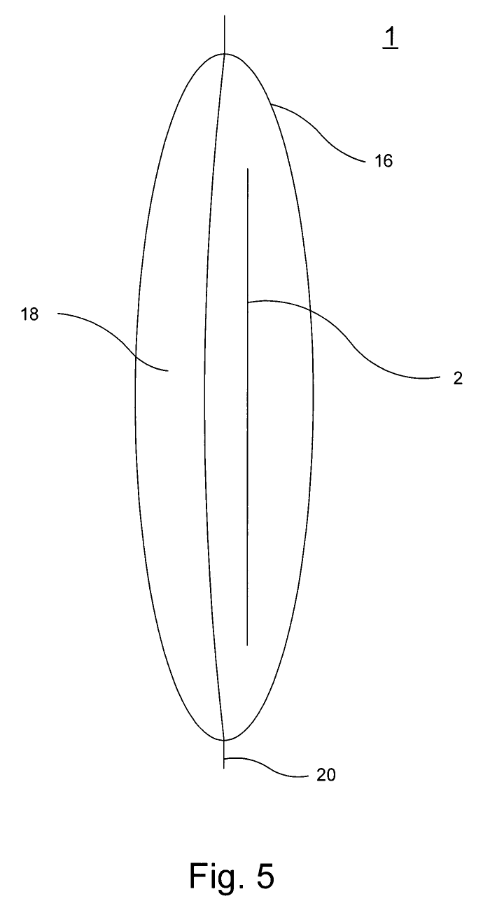

FIG. 4 shows another embodiment of the invention. As shown, a packet 1 contains a towelette 2. In one embodiment, a perforation or other opening is provided on the package for easy removal of the towelette 2. As shown in the cross-section of FIG. 4 shown in FIG. 5, the towelette 2 is in a first chamber of the packet 16, a second chamber 18 contains the disinfecting or fungicide solution. The edges of the package 20 are sealed so that each section of the packet is separate from each other. In one embodiment, the towelette 2 is in an inert atmosphere. Alternatively, the towelette 2 is pre-moistened with a first liquid, a second liquid is present in chamber 18. Prior to using the towelette, the towelette is moistened with the material in chamber 18.

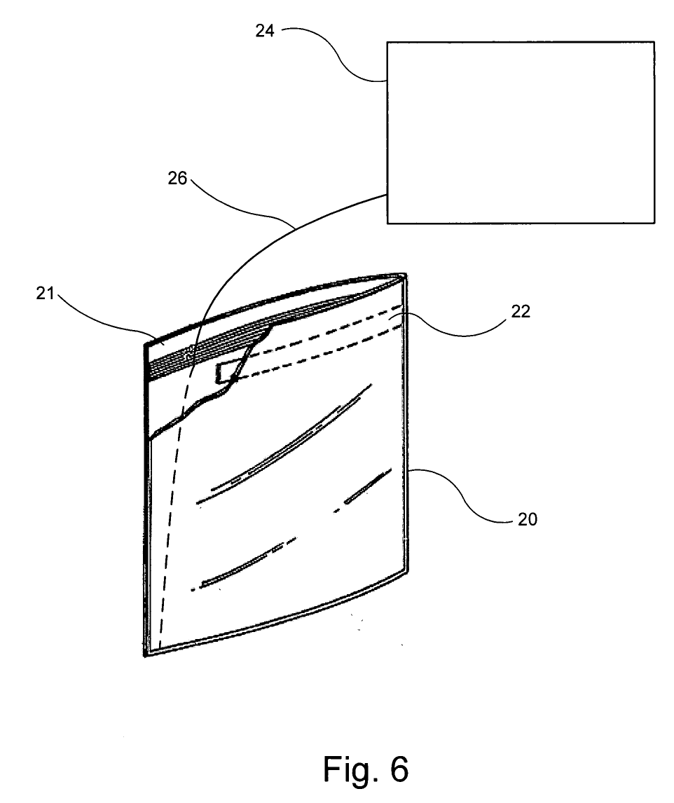

FIG. 6 shows another embodiment of the invention. As shown in FIG. 6, the packet 20 is sealed with a reusable locking mechanism 22. In another embodiment, the edge of the package 21 is sealed. Once the seal is removed, it exposes the locking mechanism. The packet can then be opened so that the moistened towelette 24 is removed. In one embodiment, the moistened towelette is attached to the package via a string or other attaching means 26 such that the packet and towelette stay connected for easy use and later disposal. The attaching means can also be elastic To use the towelette once it is moistened, either pre-moistened or the chamber containing the disinfectant and/or fungicide is ruptured thereby moistening the towelette, it is removed from the package. The tool to be cleaned is then wiped with the towelette. It should be noted that the most effective way to clean a tool is actual wiping. As discussed above, the mere dipping or spraying of a tool with a disinfecting and/or fungicide solution does not effectively remove the fungal, bacterial or viral component.

While this invention can be embodied as discussed above, it will be obvious that the invention may be varied in many ways. Such variations are not regarded as a departure from the spirit and scope of the invention, and all such modifications as would be obvious to one skilled in the art are intended to be included within the scope of the following claims.

What is claimed is:

1. A cleaning apparatus comprising:
a packet;
a towelette adapted to be placed inside the packet; and
a solution for moistening the towelette wherein the solution is at least one of a fungicide, a disinfectant, and a bactericide.

2. The packet according to claim 1 wherein a material for the packet is selected from the group consisting of mylar, plastic, foil, metalized paper, and wax paper.

3. The solution according to claim 1 wherein the solution is chosen from the group consisting of alkyl dimethyl benzyl ammonium saccharinate and ethanol, sodium hypochlorite, o-phenylphenol o-benzyl-p-chlorophenol, and quaternary ammonium chlorides.

4. The cleaning apparatus according to claim 1 wherein the solution is oil based.

5. The cleaning apparatus according to claim 3 wherein the solution is oil based.

6. The cleaning apparatus according to claim 1 further comprising at least one capsule containing the solution.

7. The cleaning apparatus according to claim 1 wherein the packet further comprises a first chamber containing the towelette and a second chamber containing the solution.

8. The cleaning apparatus according to claim 1 wherein said packet is resealable.

9. The cleaning apparatus according to claim 1 wherein said towelette is coupled to the packet.

10. A method of performing tool maintenance comprising opening a packet wherein said packet includes a pre-moistened towelette, said towelette being pre-moistened with at least one of a fungicide, bactericide, and disinfectant; and
wiping said tool with said pre-moistened towelette.

11. The method of claim 10, further comprising replacing said used towelette in said packet for disposal.

12. A method of cleaning a tool comprising:
rupturing a cell in a packet to moisten a towelette contained in the packet;
removing said moistened towelette from said packet; and
wiping said tool with said towelette.

13. The method of cleaning a tool according to claim 12 further comprising returning said towelette to said packet for disposal.

14. The method of claim 12 wherein rupturing said cell comprises breaking a capsule containing one of a fungicide, a disinfectant and a bactericide.

15. The method of claim 12 wherein rupturing said cell comprises rupturing a barrier between two portions of the packet.
